(19) United States
(12) Patent Application Publication
(10) Pub. No.: US 2006/0287612 A1
(43) Pub. Date: Dec. 21, 2006

(54) Method for simulating musculoskeletal strains on a patient

(76) Inventors: Georg N. Duda, Berlin (DE); Markus O. Heller, Berlin (DE); William R. Taylor, Berlin (DE)

Correspondence Address: THE WEBB LAW FIRM, P.C., 700 KOPPERS BUILDING, 436 SEVENTH AVENUE, PITTSBURGH, PA, 15219, US

(21) Appl. No.: 10/553,519

(22) PCT Filed: Apr. 16, 2004
(86) PCT No.: PCT/DE04/00839

§ 371 Date: Aug. 1, 2006

(30) Foreign Application Priority Data
Apr. 17, 2003 (DE) 103 18 887.8
Jul. 4, 2003 (DE) 103 31 110.6

(51) Int. Cl. A61B 5/103

(52) U.S. Cl. 600/587; 128/923

(57) ABSTRACT

The invention relates to a method and a device for simulating musculoskeletal strains on a patient. Individual musculoskeletal parameters of the patient are determined first, particularly by automatically measuring anthropometric parameters and/or the position and/or alignment of joints, especially also gait-related data such that individual musculoskeletal strains are automatically determined from the determined musculoskeletal parameters of the patient. The individual musculoskeletal strains thus determined are evaluated in a computer-assisted manner regarding at least one target criterion, particularly the contact forces or the degree of motion of a joint or the fragment movements of a fracture.

The invention relates to a method for simulating musculoskeletal strains on a patient in accordance with the preamble of claim 1.

A social trend can be seen toward an active lifestyle, marked among other things by participation in high-risk types of sports, combined with increased physical activity, even among older individuals. In addition, life expectancy is increasing, and a general aging of the population can be observed. Both phenomena are associated with an increased incidence of musculoskeletal disorders. The special relevance of these has become clear from, among other things, the Bone & Joint Decade initiated by the WHO.

In concrete terms, this means an increase in the number of people who, for example, require artificial joints, suffer fractures or require rehabilitation measures.

This development is of huge economic significance because of the associated costs of operations and rehabilitation measures or the indirect costs of incapacity for work, but it also has an important sociocultural aspect, namely that of maintaining quality of life. All of this means that optimizing the corresponding measures, for example operations and rehabilitation, is of great importance for the development of society.

However, the success of treatment, particularly in the context of joint replacement or fracture care, depends very much on the individual musculoskeletal strains existing prior to treatment and achieved thereafter. In the context of the measures concerned, it is sought to actively change these or to re-establish a “normal state”, which is generally also the optimal state. Knowledge of musculoskeletal strains is presently limited, because these involve complex systems. Musculoskeletal strains are presently not included in the planning or conduct of operations and/or rehabilitation measures. The result of the operation performed or of the rehabilitation measures is therefore to a large extent dependent on the experience of the surgeon or of the therapist.

It is therefore an object of the present invention to make available a method for evaluating musculoskeletal strains on a patient, with which method it is possible in particular to improve surgical interventions and rehabilitation measures. The object is achieved by a method for simulating musculoskeletal strains having the features of claim 1.

Accordingly, individual musculoskeletal parameters of the patient are first determined. In particular, by automatic measurement, anthropometric parameters are determined, automatic derivation of anthropometric parameters from a system for computer-assisted surgery, particularly a surgical navigation system, and/or the position and/or orientation of joints. A surgical navigation system provides the operating physician with a virtual display of the operating area. This display is created, for example, on the basis of CT images recorded prior to the operation. The operating physician is also able to observe the movements of instruments in the virtual display and can follow a treatment plan introduced beforehand in the virtual display. Comparison of the actual operating situation with the planned situation is thus also possible. It is also possible to actively control instruments with the aid of the navigation system. The method and the device for carrying out the method can, for example, be installed on a central server. The users (e.g. therapists, surgeons, technicians) would in this case not access data locally, but could instead obtain, or if appropriate also input, the required information over the Internet. The data can thus be utilized to accompany the therapy during the planning stage and also in subsequent stages. A center can also be set up to centrally manage and distribute data for different users.

In the second step, the individual musculoskeletal strains are determined automatically from the determined individual musculoskeletal parameters. In the third step of the method according to the invention, the individual musculoskeletal strains are evaluated, in a computer-assisted manner, in respect of at least one target criterion. The target criterion used can include, in particular, the contact forces or the degree of movement of a joint or the fragment movements of a fracture.

The method according to the invention can be used to assist in surgical procedures, for example in interventions for total joint replacement, interventions on ligament structures, interventions in the context of displacement osteotomies, and interventions in the context of fracture management in humans and animals.

By means of the determination of an evaluation of the individual musculoskeletal strains in respect of at least one target criterion, surgical methods can be assisted in terms of their planning, performance and evaluation. With the aid of the method, the surgeon or therapist can directly assess the effects of a planned intervention in a noninvasive manner and can accordingly evaluate and adapt his operating plan or therapy plan, respectively. Through the use of individualized, biomechanical musculoskeletal strain and load analyses, the planning, performance and evaluation of surgical methods can therefore already be objectivized before the intervention.

In an alternative embodiment of the method, at least one of the musculoskeletal parameters, particularly the position and/or orientation of a joint, is varied following evaluation of the individual musculoskeletal strains. Thereafter, the individual musculoskeletal strains are again automatically determined taking into consideration the at least one varied musculoskeletal parameter. This is followed once more by computer-assisted evaluation for individual musculoskeletal strains in respect of the at least one target criterion. In this way, a comparison can be made between two possible situations or operating plans. For example, it is possible to investigate the effect of a different position of a joint in respect of the at least one target criterion, for example in respect of the arising contact forces of the joint. Precise planning of an operation is made possible in this way. In a further development of the invention, the varied parameter can be optimized by repeating the variation of the at least one parameter until a specified target value of at least one target criterion is reached. In this way, optimization of the target criterion and thus also of the set of parameters is achieved in an iterative procedure. In this way, for example, the optimal position of an artificial joint can be determined.

The musculoskeletal parameters thus determined, for example the position of a joint, are advantageously output on an output unit and/or stored in a storage unit. In addition, or alternatively, the output data can also be transferred to a computer-assisted surgery system and/or to a surgical navigation system, so that these data can also be made available during an operation.

The individual and varied musculoskeletal parameters obtained in this iterative procedure and corresponding to the target value advantageously serve as a basis for planning a surgical intervention. They particularly serve as a basis for the choice of components, for example different joint types, with regard to the positioning of components or the decision regarding the removal of temporary implants.

To be able to determine the effects of different implants on the musculoskeletal strain on the patient, the variation of the individual musculoskeletal parameters can be carried out taking into consideration the data for implants, particularly their dimensions and ranges of movement. Thus, for example, different implants can be tested against one another, and the optimal implant for the particular patient anatomy can be selected taking into consideration the respective target criteria.

In developments of the method according to the invention, two different types of method are provided for the automatic determination of the individual musculoskeletal strains:

On the one hand, the individual and the varied musculoskeletal parameters are compared with musculoskeletal reference parameters filed in a database, and musculoskeletal reference strains corresponding to the musculoskeletal reference parameters are determined as the individual musculoskeletal strains. The musculoskeletal reference parameters can in this case be present as discrete values in the database. In the presence of discrete values, it is recommended to compare the reference parameters with the individual musculoskeletal parameters by means of functional relationships, particularly by means of interpolation.

On the other hand, in a development of the method according to the invention, the individual musculoskeletal strains are calculated from the determined individual musculoskeletal parameters. The calculation is advantageously based on a biomechanical and/or a mathematical model. In a development of the method according to the invention, the respectively used biomechanical and/or mathematical model is already adapted to the individual musculoskeletal parameters. For this purpose, a biomechanical and/or a mathematical model can be chosen on the basis of the determined individual musculoskeletal parameters from at least one database.

The chosen model is then optimized and adapted to the determined individual musculoskeletal parameters. Therefore, the individual musculoskeletal strains are advantageously calculated with the aid of a musculoskeletal model taking into consideration the individual patient anatomy or taking into consideration the patient's individual anthropometric data.

To simplify the computer-assisted evaluation of the respective results of the method, these individual musculoskeletal strains are advantageously visualized. On the basis of the visualization, the attending physician or therapist can quickly and easily check and alter his treatment plan. The individual musculoskeletal strains are in this case advantageously presented on the basis of an anatomical model, in graph form and/or numerically.

Through the evaluation of the individual musculoskeletal strains, a rehabilitation process can also be evaluated and/or managed, for example being able to be recognized. Thus, for example, corresponding data can be accessed via the Internet.

In an advantageous development of the method, the individual musculoskeletal parameters of the patient are determined by measurements. To simplify the method and make it more objective, at least one of the individual musculoskeletal parameters can be measured automatically. Such a measurement can take place particularly by image analysis, computed tomography and/or by motion sensors.

It is also advantageous if individual movement parameters, particularly gait parameters, are determined, and these are used for the automatic determination of individual musculoskeletal strains. For example, individual gait parameters can be obtained by recording images of the legs in motion. Three-dimensional positions of the parts of the body can then be determined from the images. The reaction force which acts on the foot from the floor or ground is also measured. It is particularly advantageous if the individual gait parameters are determined from personal data stored in a database and/or are recorded individually for one person.

The object is also achieved by a device having the features of claim 22. Such a device can be implemented as a software-assisted and/or hardware-assisted variant in a data-processing installation. This data-processing installation then has a connection to a database with which musculoskeletal strains and/or individual movement parameters can be stored.

The method is explained in more detail below with reference to the drawings of the figures, in which:

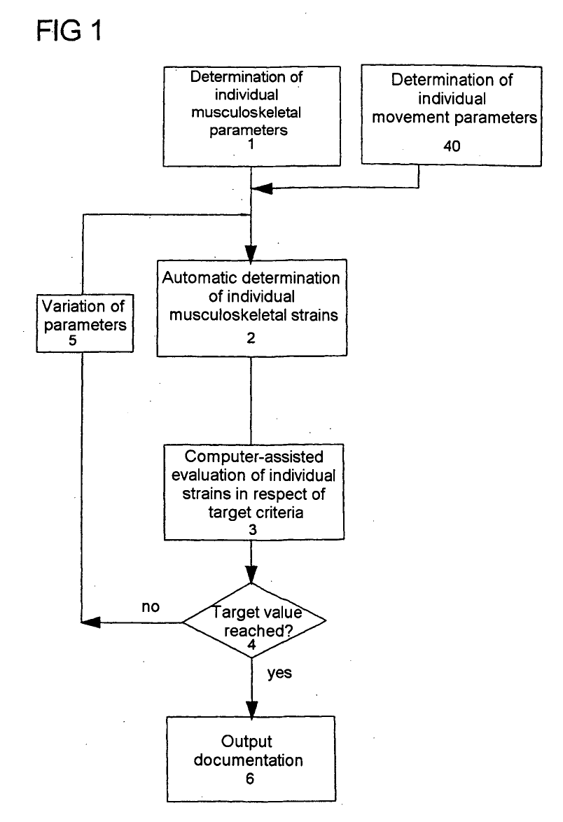

FIG. 1 shows a schematic representation of the method according to the invention in a first embodiment;

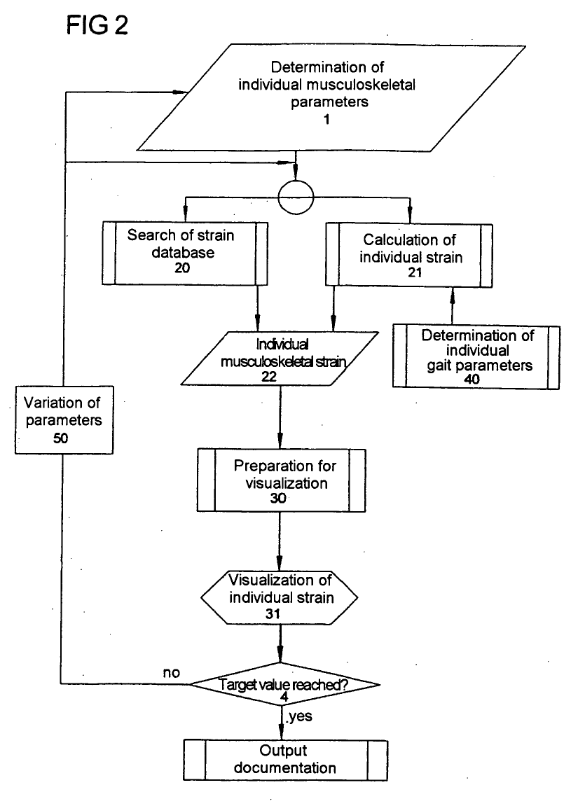

FIG. 2 shows the method in a second, detailed embodiment;

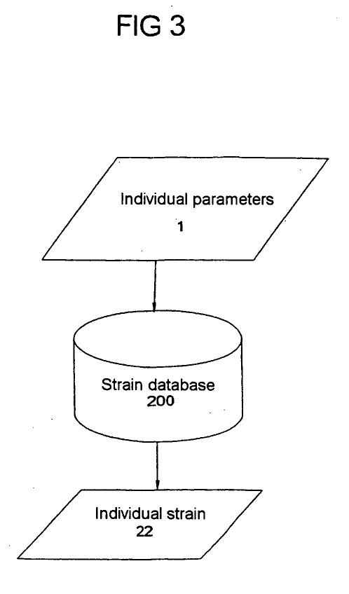

FIG. 3 shows a section from the method shown in FIG. 2, concerning the determination of individual musculoskeletal strains from a database;

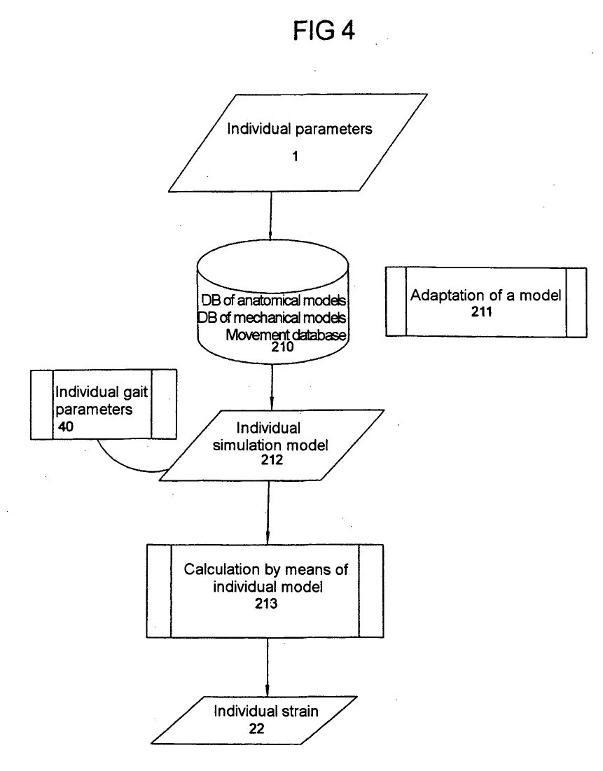

FIG. 4 shows a detailed section from the method in FIG. 2, illustrating the calculation of musculoskeletal strains;

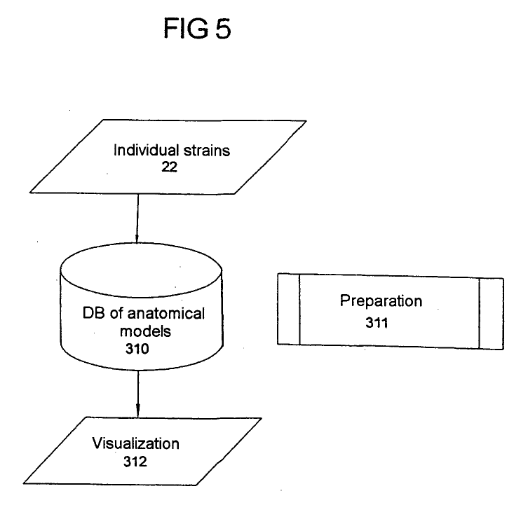

FIG. 5 shows a further section from the method in FIG. 2, concerning visualization; and

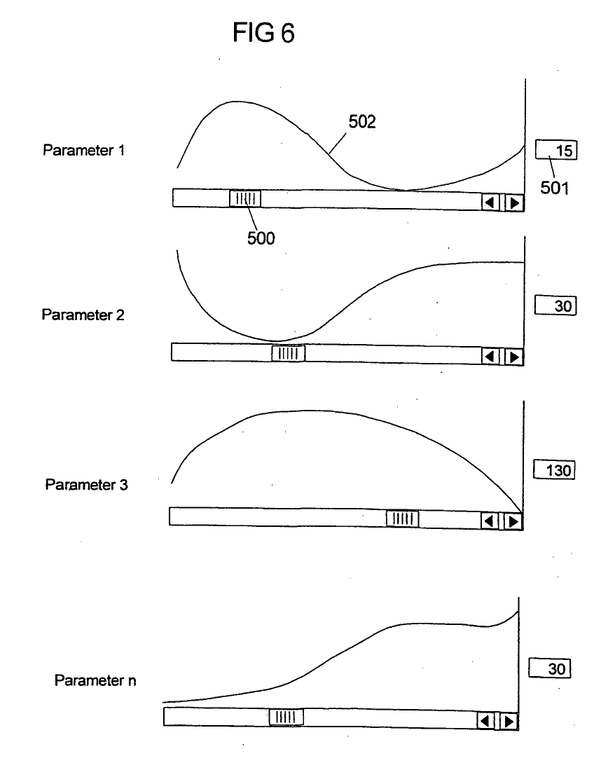

FIG. 6 shows a representation of a possible visualization of musculoskeletal strains.

The method according to the invention is shown schematically in FIG. 1. In the first step, the individual musculoskeletal parameters of the particular patient are determined. These particularly include anthropometric data, such as the bone measurements and their densities, the points of gravity of the bones and other inertia parameters, the pelvis dimensions, the respective lengths of the thigh and lower leg, or the foot length. Other anthropometric data may also be included which are associated with the respective automatic determination of the individual musculoskeletal strains.

The determination of the individual musculoskeletal parameters in step 1 can be determined by automatic measurements, which are taken for example from computed tomography, by external measurements of the patient, by movement analyses or by other measurement methods. In addition, or alternatively, anthropometric parameters can be taken over automatically from a navigation system.

In step 40, for example, individual movement parameters, can be introduced into the method. In the case of the lower limbs, a gait analysis can be used for this purpose. Here, the movements of the individual leg segments are recorded, for example by an optical measurement system, during certain activities (e.g. walking, climbing stairs, standing up from a chair, bending the knees, etc.). To permit the optical recording, reflecting markers that can be recorded by the measurement system are placed on a patient. In this way, the spatial and chronological position of the body segments (e.g. pelvis, thigh, knee, lower leg, foot) can be determined. In conjunction with a number of cameras, a three-dimensional movement image of the body segments can be obtained from the two-dimensional images. The reaction force of the floor or ground on the feet is also measured in the gait analysis. The movement parameters can be calculated from a patient's database values (e.g. height, weight) and/or can be recorded directly from the patient. Although gait parameters are focussed on in the text below, it is in principle also possible to use movement parameters of other parts of the body.

In step 2, the individual musculoskeletal strains are automatically determined from the determined individual musculoskeletal parameters and if appropriate from the individual movement parameters. This determination can be done, for example, by comparing the determined musculoskeletal parameters with reference parameters filed in a database, or by calculating the respective individual musculoskeletal strains. These methods are explained in more detail in the description of the following figures.

In step 3 of the method shown in FIG. 1, the automatically determined individual musculoskeletal strains are now evaluated in a computer-assisted manner in respect of a target criterion. The target criterion can, for example, be the contact forces or the degree of movement of a joint or the necessary fragment movements of a fracture. A combination of several target criteria may also be considered and evaluated.

In a simple embodiment of the method, the evaluation and the individual strains are then output or documented in step 6.

To optimize a set of parameters, it is also possible, in a development of the method, to check in step 4 whether the target criterion has reached a predefined target value. If this is not the case, at least one of the individual parameters is varied in step 5. The automatic determination of the individual strains in step 2 and the evaluation of these individual strains in respect of a target criterion in step 3 follow on from this. If the target value in step 4 is not yet reached, at least one parameter is once again varied in step 5. By contrast, if the target value is reached, output and documentation take place in step 6. It is also possible for the data to be output automatically to a surgical navigation system.

By means of this iterative procedure, a set of parameters can be optimized in respect of one or more target criteria. The individual set of parameters that has been optimized in respect of the individual musculoskeletal strains or in respect of the target criterion can then be used to plan a surgical intervention, for example, or to plan therapeutic measures.

The variation of the at least one parameter in step 5 of the figure can also take into consideration the dimensions or other parameters of implants. Thus, through the variations of the parameters in step 5, different implants can be tested against one another.

The data obtained and processed are stored and can be used for rehabilitation. For example, the physician concerned can access the data via the Internet in order to set up a specially adapted rehabilitation plan, for example for physiotherapy.

FIG. 2 shows the method according to the invention in an extended embodiment. First, in step 1, the individual musculoskeletal parameters of the respective patient are determined, for example by automatic measurement. The automatic determination of the individual musculoskeletal strains from the determined individual musculoskeletal parameters can then be done either in step 20 by searching a database or in step 21 by calculating the individual musculoskeletal strain. The individual gait parameters are also included (step 40).

From step 20 or step 21, the individual musculoskeletal strain of the particular patient is then available in step 22. The individual musculoskeletal strain is now prepared for visualization in step 30. The way in which this is done is set out in detail in the description of the following figures.

In step 31, the respective individual musculoskeletal strains are visualized. From this visualization, the musculoskeletal strains are now evaluated in a computer-assisted manner to ascertain whether at least one target criterion has reached a predefined target value. If this is the case in step 4, the corresponding parameters and the musculoskeletal strains are output and documented in step 6. If the target value is not reached in step 4, at least one parameter is changed or varied in step 50 on the basis of the visualization.

With the new set of parameters generated in step 50, an automatic determination of the individual musculoskeletal strains is again carried out in step 20 or step 21. This iterative process is continued until the target value of at least one target criterion is reached in step 4.

The automatic determination of the individual musculoskeletal strains in both said alternatives is shown now in FIGS. 3 and 4.

In FIG. 3, the individual musculoskeletal parameters determined in step 2 are compared with reference parameters filed in a strain database 200. The reference parameters in the strain database 200 can be present either as discrete values or as continuous values.

In the presence of discrete values, the comparison between the determined individual musculoskeletal parameters and the reference parameters in the strain database is carried out via functional relationships, in particular via interpolation. The reference strains corresponding to the reference parameters closest to the determined parameters are then output as the individual musculoskeletal strain in step 22.

In this alternative, before carrying out the method according to the invention, it is necessary to record a greater quantity of empirical data, on the basis of which the strain database 200 can be constructed.

FIG. 4 shows the second alternative of the automatic determination of the musculoskeletal strains. From the determined individual musculoskeletal parameters in step 1, a suitable anatomical mechanical and/or biomechanical model or a suitable movement model is to be selected from a database 210.

The selected model is adapted in step 211 to the determined individual musculoskeletal parameters 1. In step 212, this gives an individual biomechanical model which is adapted individually to the particular patient. Individual gait parameters 40 are also included here.

With the individual biomechanical model, the individual musculoskeletal strains are now calculated in step 213. These individual musculoskeletal strains are then assigned to the individual musculoskeletal strain in step 22.

FIG. 5 shows the individual method steps for visualizing the individual musculoskeletal strains. The individual musculoskeletal strains from step 22 are prepared together with the data from a database 310 of an anatomical model in step 311. In this case, for example, the individual musculoskeletal strains can be assigned to individual anatomical parts. Data can also be processed in a surgical navigation system. The data that have been linked and prepared in this way are visualized in step 312.

Such visualization is shown by way of example in FIG. 6. Individual parameters 1 to m can be varied on the basis of the visualization, for example on the basis of a graph representation, on a computer screen with the aid of a slide 500. The value of the respective parameter is shown in a separate display 501. This can, for example, involve an angle or a distance. The musculoskeletal strain is visualized on the basis of a curve 502 which shows the strains during a walking cycle, for example, or while climbing stairs.

The attending physician or the therapist is now able, by moving the slide 500, to set different values of the respective parameter and to see how the strain data change.

This visualization and the simultaneous changing of the parameters permits rapid and efficient pinpointing of optimal solutions and planning of an intervention. The attending physician or therapist can therefore see at a glance what consequences a change of a parameter has in respect of a target criterion.

The description of the figures relates to a specific joint, namely a hip joint. The teaching according to the invention, however, can in principle be applied to all joints, in particular knee joints, shoulder joints, ankle joints, jaw joints, elbow joints and/or vertebral joints.

1-25. (canceled)

26. A method for simulating musculoskeletal strains on a patient for monitoring surgical interventions, the method comprising the steps of:
a. determining individual musculoskeletal parameters of the patient and automatically deriving anthropometric parameters from a system for computer-assisted surgery;
b. automatically determining the individual musculoskeletal strains from the determined musculoskeletal parameters of the patient; and
c. for the automatic determination of the individual musculoskeletal strains, comparing the individual and varied musculoskeletal parameters with musculoskeletal reference parameters filed in a strain database constructed with empirical data, and musculoskeletal reference strains corresponding to the musculoskeletal reference parameters are determined as the individual musculoskeletal strains, the musculoskeletal reference parameters being present as discrete values in the strain database and the musculoskeletal reference parameters being compared with the individual musculoskeletal parameters by means of functional relationships; and
d. evaluating the individual musculoskeletal strains in respect of at least one target criterion.

27. The method as claimed in claim 26, further comprising the steps of:
e. varying at least one musculoskeletal parameter;
f. subsequently automatically determining the individual musculoskeletal strains taking into consideration the at least one varied musculoskeletal parameter; and
g. subsequently evaluating the individual musculoskeletal strains in respect of the at least one target criterion.

28. The method as claimed in claim 27, wherein steps e. to g. are repeated until a specified target value of at least one target criterion is reached.

29. The method as claimed in claim 28, wherein the musculoskeletal parameters corresponding to the target value are output on an output unit, stored in a storage unit and/or transferred to a computer-assisted surgery system and/or to a surgical navigation system.

30. The method as claimed in claim 28, wherein the individual and varied musculoskeletal parameters corresponding to the target value serve as a basis for planning a surgical intervention, the positioning of components or the decision regarding the removal of temporary implants.

31. The method as claimed in claim 27, wherein the variation of the individual musculoskeletal parameters in step e. is carried out taking into consideration predefinable data for implants.

32. The method as claimed in claim 26, wherein the individual musculoskeletal strains are calculated from the determined individual musculoskeletal parameters.

33. The method as claimed in claim 32, wherein a biomechanical and/or a mathematical model is used as a basis for the calculation of the individual musculoskeletal strains.

34. The method as claimed in claim 33, wherein the biomechanical and/or mathematical model is adapted to the individual musculoskeletal parameters.

35. The method as claimed in claim 33, wherein the biomechanical and/or mathematical model is chosen on the basis of the determined individual musculoskeletal parameters from at least one database.

36. The method as claimed in claim 34, wherein the individual musculoskeletal strains are calculated with the aid of a musculoskeletal model taking into consideration the individual patient anatomy.

37. The method as claimed in claim 26, wherein the individual musculoskeletal strains are visualized for evaluation.

38. The method as claimed in claim 26, wherein the individual musculoskeletal strains are presented on the basis of an anatomical model, particularly in graph form and/or numerically.

39. The method as claimed in claim 26, wherein, by evaluation of the individual musculoskeletal strains, a rehabilitation process is evaluated and/or managed, particularly by means of Internet access.

40. The method as claimed in claim 26, wherein the individual musculoskeletal parameters of the patient are determined by measurements.

41. The method as claimed in claim 40, wherein at least one of the individual musculoskeletal parameters is measured automatically.

42. The method as claimed in claim 26, wherein individual movement parameters, particularly gait parameters, are determined, and these are used for the automatic determination of individual musculoskeletal strains.

43. The method as claimed in claim 42, wherein the individual gait parameters are determined from personal data stored in a database and/or are recorded individually for one person.

44. The method as claimed in claim 26, wherein the position and/or orientation of joints are used for a navigation system for computer-assisted surgery and/or the data from a navigation system are used for computer-assisted surgery.

45. A device for evaluating musculoskeletal strains on a patient, with means for carrying out the method as claimed in claim 26.

46. A movement analysis system coupled to the device as claimed in claim 45.

47. A navigation system for computer-assisted surgery for carrying out the method as claimed in claim 26.

48. The method as claimed in claim 26, wherein the musculoskeletal parameters are automatically measured anthropometric parameters.

49. The method as claimed in claim 26, wherein the target criterion include contact forces, degree of joint movement, fragment movements of a fracture or any combination thereof.
